(19) United States
(12) Patent Application Publication
(10) Pub. No.: US 2011/0282445 A1
(43) Pub. Date: Nov. 17, 2011

(54) Transport device for ossicular prosthesis

(76) Inventors: Uwe Steinhardt, Hirrlingen (DE); Walter Gamer, Stutensee (DE)

(21) Appl. No.: 13/103,679

(22) Filed: May 9, 2011

(30) Foreign Application Priority Data
May 12, 2010 (DE) 20 2010 006 737.5
Jun. 24, 2010 (DE) 10 2010 024 894.0

(51) Int. Cl. A61F 2/18

(52) U.S. Cl. 623/10

(57) ABSTRACT

A transport device for receiving and securely holding an ossicular prosthesis for implantation in a middle ear during transport to an operating surgeon for replacing or bridging at least parts of a component of a human ossicular chain, the transport device has a flat base plate of thin metal sheet having a thickness of substantially half a millimeter, at least one suspending device connected to the base plate and twistable using fine segments and integrated in the base plate for receiving and holding the ossicular prosthesis, and the connecting element which connects a first fastening element of the ossicular prosthesis used for mechanical connection to a tympanic membrane or a component of the ossicular chain with a second fastening component on another end used for mechanical connection to a further component or parts of a component of the ossicular chain or directly to an inner ear so that the first and second fastening elements are connected by the connecting element in a sound-conducting manner, wherein the suspending device is configured so that it aligns with the surface of the base plate during manufacture of the transport device and for suspension of the ossicular prosthesis is rotatable relative to an ossicular prosthesis lying-flat production state by substantially 90° relative to the base plate by plastic deformation of the segments and then extends above the surface of the base plate.

### CROSS-REFERENCE TO RELATED APPLICATION

The invention described and claimed hereinbelow is also described in German Patent Applications DE 20 2010 006 737.5 filed on May 12, 2010 and DE 10 2010 024 894.0 filed on Jun. 24, 2010. These German Patent Applications, whose subject matter is incorporated here by reference, provides the basis for a claim of priority of invention under 35 U.S.C. 119(a)-(d).

### BACKGROUND OF THE INVENTION

The invention relates to a transport device designed to receive and securely hold an ossicular prosthesis for implantation in the middle ear during transport to the operating surgeon, wherein the ossicular prosthesis can replace or bridge at least parts of a component of the human ossicular chain.

Such a transport device for ossicular prosthesis is known from U.S. Pat. No. 4,288,066.

Ossicular prosthesis are used to conduct sound or the sound signal from the tympanic membrane to the inner ear in cases in which the ossicles of the human middle ear are missing or damaged, in entirety or in part. The ossicular prosthesis has two ends. Depending on the specific circumstances, one end of the ossicular prosthesis is fastened to the tympanic membrane, e.g. using a top plate, and the other end of the ossicular prosthesis is fastened, e.g. to the stapes of the human ossicular chain, or it is inserted directly into the inner ear. Three types of ossicular prosthesis that are used particularly frequently are stapes prosthesis, partial prosthesis, and total prosthesis. Stapes prosthesis are fixed to the incus, and extend via a piston into the inner ear. Partial prosthesis typically bear via a top plate against the tympanic membrane and establish a connection to the head of the stapes. Total prosthesis connect the tympanic membrane to the base of the stapes.

Since ossicular prosthesis naturally have outer diameters in the range of a few millimeters, and are very fragile in terms of the functional fine structure thereof, transport devices of the type in question, which are used to transport such fragile prosthesis from the manufacturer to the operating surgeon, must fulfill the hygiene requirements on a sterile packaging and ensure that the particular prosthesis arrives undamaged.

A feasible option therefor would be to use holders for parts to be milled, or injection-molded parts composed of solid material which have cavities of suitable shape and size for accommodating the ossicular prosthesis for transport, but which result in relatively high manufacturing costs.

Document U.S. Pat. No. 5,669,501 makes known a transport device for an ossicular prosthesis, which comprises a flat base plate in the form of a bottom wall of an insert into which a suspending device for holding the ossicular prosthesis is integrated.

Initially cited document U.S. Pat. No. 4,288,066 describes a container for a middle ear prosthesis, which is intended to be suitable for transporting the prosthesis to the operating surgeon and for the mechanical preparation thereof by the operating surgeon. This known transport device is composed of a relatively thick, injection-molded container made of plastic and comprising a suitable recess for insertion of the prosthesis, and a cover for closing the container, which is closably attached to the container by way of a tab. The recess is designed such that the middle ear prosthesis can be cut to a desired length before implantation, even before removal thereof from the container.

The disadvantages of this known container are the relatively bulky—relative to the size of the prosthesis—dimensions thereof, the complicated internal design thereof, the resultant costs for the corresponding production tool, and the fact that the geometry predefined by the injection-molding tool for the container results in only one certain shape and size for the particular prosthesis.

### SUMMARY OF THE INVENTION

The problem addressed by the present invention is thus that of providing a transport device for ossicular prosthesis of the type described initially, using the simplest possible technical means, and in a simple and economical manner, wherein the transport device is very lightweight and has the most compact dimensions possible, is easily manufactured and allows for great flexibility in terms of sizes and geometries of the middle ear prosthesis to be transported, wherein a secure hold of the prosthesis during transport is ensured, and the prosthesis can be prepared by the operating surgeon conveniently within the transport device itself before removal.

This problem is solved according to the invention in a manner that is surprisingly simple and effective in that the transport device comprises a flat base plate of thin metal sheet having a thickness of approximately half a millimeter, into which at least one, preferably two suspending devices—which are connected to the base plate and can be twisted using fine segments—are integrated for receiving and holding the ossicular prosthesis, wherein the ossicular prosthesis comprises, on one end thereof, a first fastening element for mechanical connection to the tympanic membrane or a component of the ossicular chain, and, on the other end thereof, a second fastening element for mechanical connection to a further component or parts of a component of the ossicular chain, or directly to the inner ear, and a connecting element which connects the two fastening elements in a sound-conducting manner, and wherein the suspending device aligns with the surface of the base plate during manufacture of the transport device, and, for suspension of the ossicular prosthesis, can be rotated relative to this lying-flat production state by approximately 90° relative to the base plate by way of plastic deformation of the segments, and then extends above the surface of the base plate.

The transport device according to the invention can be manufactured with outer dimensions that are barely larger than those of the ossicular prosthesis to be accommodated, wherein the weight of the device—depending on the starting material that is selected—is likewise within the same order of magnitude of the weight of the prosthesis. The swivellable suspending devices can be easily designed without considerable production outlay such that middle ear prosthesis of different sizes and geometries can also be securely accommodated and held, wherein the rotation by approximately 90° relative to the base plate after transport and before removal of the prosthesis enables preparation to be carried out in an ergonomically favorable way.

Particularly preferably, the transport device according to the invention is manufactured by machining the base plate using laser treatment and/or anodizing with high accuracy of the fine structures to be created.

Embodiments of the invention in which the base plate and the suspending device are both fabricated out of one integrated blank can be manufactured in a very particularly favorable manner.

One class of further advantageous embodiments of the invention is characterized by the fact that the transport device comprises an outer packaging which encloses the base plate on all sides during transport, and is in the form of a container in particular, such as a case, a can, or a box.

In regard to transporting the ossicular prosthesis without damage, developments of these embodiments are advantageous in which the base plate comprises a device for ensuring safe transport, which has burr-shaped segments extending laterally outwardly from the base plate, and can be clamped or braced against inner walls of the outer packaging.

The fastening of ossicular prosthesis—for which the transport device of the type in question type is provided—can be brought about or at least supported on a component of the ossicular chain during implantation by manufacturing the entire prosthesis, or at least the corresponding fastening element, of a material having memory effect—typically the nickel-titanium alloy Nitinol—and which was subjected preoperatively to deformation under defined temperature conditions. In order to now permanently attach a fastening element, which is designed e.g. as a loop, the loop is initially placed relatively approximately around the miniscule subregion of the ossicle, and is then positioned exactly and subjected to thermal treatment—usually using a laser or an electrical device—such that the specific deformation of the memory material results in a final closure of the loop around the ossicle, said closure becoming permanent at body temperature. An analogous procedure is used to fasten elements having other shapes.

In that particular case it is problematic that the application of excessive luminous power or electrical energy during implantation of the middle ear prosthesis has the undesired effect of excessive local heating and, therefore, generating a temperature that is too high in the region of the fastening element, which is transmitted to the ossicle and can quickly result in damage or even necrosis of the sensitive bodily tissue at the corresponding section of the ossicle. The final result of the entire operation could thus be counterproductive.

Of course the manufacturers of middle ear prosthesis of the type in question typically make claims about optimal method parameters for the “preparation” of their prosthesis having memory effect. However, due solely to the fact that the devices employed on-site by the users for thermal treatment can differ greatly, these claims can only cover ranges that are very wide and are therefore often of little informative value. In addition, it does little good to inform an operating surgeon about what local maximum temperature he must not exceed in the thermal treatment. He certainly knows that protein coagulates at temperatures starting at approximately 60° C. and can permanently damage bodily tissue. Thus he must find a suitable setting for the power output of the device he will use, and identify the point of energy application onto the ossicle prosthesis that is optimal for his purposes, before he performs the operation in the middle ear.

To provide the operating surgeon with an uncomplicated way to determine a parameter setting—intraoperatively and shortly before the actual implantation of the middle ear prosthesis—that is optimal in terms of the thermal treatment devices available to him, without any need for the middle ear prosthesis to be touched and therefore possibly changed in any way, which is undesired, it is advantageous to provide a device for determining an optimized power setting and/or energy application site for preparing the components of the ossicle prosthesis, which are to be subjected intraoperatively to thermal treatment, the device containing a prosthesis mock-up which matches the prosthesis and is identical to the ossicular prosthesis in terms of material, geometric shape, and manufacturing method, at least in the regions in which the associated prosthesis will be subjected intraoperatively to thermal treatment.

In this manner, the operating surgeon can determine the optimal parameters for the thermal treatment to be performed in situ ^{on} the ossicular prosthesis in the middle ear in advance on the prosthesis mock-up using the devices available to him, and easily test out planned handling steps on the outside instead of in the middle ear, without any need to even touch the original prosthesis and thereby possibly cause deformation or deformation of same. The parameters determined in this manner correspond very exactly to the parameters that apply for the original prosthesis, because the mock-up of the prosthesis is an identical match to the original prosthesis, at least at the points intended for thermal treatment.

Proceeding therefrom, a class of embodiments of the transport device according to the invention is very particularly preferred and is characterized in that the transport device comprises a holding device which can also swivellably accommodate the above-described mock-up of the prosthesis.

Developments of these embodiments that are particularly preferred and are convenient for the user are characterized in that the holding device for the prosthesis mock-up contains another holding device for the attachment of one end of the prosthesis mock-up, which is connected to the base plate and can be twisted using fine segments. It is therefore possible to ensure that the prosthesis mock-up is positioned in a stable, easily accessible manner while the optimal treatment parameters are being determined, and that transport to the “application site” is gentle and damage-free.

A further considerable improvement in terms of handling is attained in variants of the above-described embodiments, in the case of which the prosthesis mock-up contains a detent device which can be used to secure the prosthesis mock-up in the base plate, at least in a processing position. The rigid and non-displaceable position of the prosthesis mock-up during determination of optimal parameters simplifies the work being performed and increases the quality of the parameters that are found.

To protect the particularly sensitive sections of the middle ear prosthesis and the associated prosthesis mock-up, place-holder pins are provided in preferred developments, which can be inserted into the base plate or are integrated in the base plate, and which are formed such that they affix the thermally pretreated parts of the ossicular prosthesis composed of material having memory effect, and optionally the corresponding sections of the associated prosthesis mock-up in the desired geometry during transport to the operating surgeon such that unwanted changes in shape are prevented even if warming occurs during transport.

Embodiments of the transport device according to the invention are also particularly preferred in which the base plate is optically coded and/or labeled, wherein the coding or labeling contains technical information regarding the ossicular prosthesis and/or an associated prosthesis mock-up and/or manufacturer's information, thereby eliminating the need to look up these important data in a manual or information sheet provided with the transport device, since they are available directly during handling of the transport device.

Finally, developments of these embodiments are of particular practical advantage in which the technical information of the coding and/or labeling contains a recommended starting value for the electrical power or luminous power, which should be used within the scope of a test handling of the prosthesis mock-up at the beginning of the determination of optimized processing parameters for the components of the ossicular prosthesis to be subjected intraoperatively to thermal treatment.

Further features and advantages of the invention will become apparent from the detailed description of embodiments of the invention presented below with reference to the figures in the drawing which shows the details that are essential to the invention. Further features and advantages of the invention will also become apparent from the claims. The individual features may be implemented individually, or they may be combined in any possible manner to form variants of the invention.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 shows an embodiment of the transport device according to the invention, which comprises an associated ossicular prosthesis and, adjacent thereto, a prosthesis mock-up swivellably accommodated on a holding device in the region of a detent device, in a schematic spacial depiction at an angle from above;

FIG. 2a shows a detailed view of the prosthesis mock-up in the region of the detent device in the embodiment depicted in FIG. 1;

FIG. 2b shows a detailed view of the entire prosthesis mock-up in a schematic spacial depiction at an angle from above, rotated by approximately 180° relative to FIG. 1;

FIGS. 3a-3e show the embodiment depicted in FIG. 1, in a schematic horizontal projection
(a) from above,
(b) from below,
(c) of an end face I,
(d) of opposite end face II, and
(e) of one longitudinal side III;

FIG. 4a shows the embodiment in FIG. 1 in a schematic spacial depiction at an angle from above with ossicular prosthesis removed, and with prosthesis mock-up upright and locked in position with the holding device;

FIG. 4b is the same as FIG. 4a, but rotated by approximately 180° from the perspective of the opposite longitudinal side;

FIG. 5a shows the base plate of the embodiment depicted in FIG. 1 without ossicular prosthesis and without prosthesis mock-up, and with the swivellable holder for the prosthesis and the suspending device for the mock-up, each in the unswivelled, lying-flat production state;

FIG. 5b shows the transport device depicted in FIG. 5a in a schematic view from below;

FIG. 5c shows the transport device depicted in FIG. 5a with the suspending device for the ossicular prosthesis in the swivelled state for receiving the prosthesis to be transported; and

FIG. 5d shows the transport device depicted in FIG. 5c in a schematic view from below.

### DESCRIPTION OF THE PREFERRED EMBODIMENTS

The embodiment of transport device 1 according to the invention—which is depicted schematically in the figures of the drawing and includes an ossicular prosthesis 2 for implantation in the middle ear, which comprises on one end thereof a first fastening element 2a for mechanical connection to the tympanic membrane or a component of the ossicular chain, and, on the other end thereof, a second fastening element 2b for mechanical connection to a further component of the ossicular chain or directly to the inner ear, and a connecting element 2c which connects the two fastening elements 2a, 2b in a sound-conducting manner, wherein at least parts of ossicular prosthesis 2 are made of a material having memory effect and are subjected to shape-altering thermal treatment when ossicular prosthesis 2 is implanted in the middle ear—is characterized, according to the invention, in that transport device 1 comprises a flat base plate 5 which is typically formed of a thin metal sheet having a very small thickness, and into which at least one suspending device 6b is integrated, is connected to base plate 5, can be twisted by way of fine segments 6b′, and is used to receive and hold ossicular prosthesis 2, and in that suspending device 6b aligns with the surface of base plate 5 during manufacture of transport device 1, and, for suspension of ossicular prosthesis 2, can be rotated relative to this lying-flat production state by approximately 90° relative to base plate 5 by way of plastic deformation of segments 6b′, and then extends above the surface of base plate 5.

Advantageously, transport device 1 is manufactured together with suspending device 6b out of one integrated blank by machining base plate 5 using laser treatment and/or anodizing.

Furthermore, transport device 1 according to the invention also contains a prosthesis mock-up 3 which is designed to match ossicular prosthesis 2 and is part of a test device for determining an optimized power setting and/or energy application site for the preparation of parts of ossicular prosthesis 2 to be subjected intraoperatively to thermal treatment. Prosthesis mock-up 3 is identical in design to ossicular prosthesis 2 in terms of material, geometric shape, and manufacturing method, at least in the regions in which associated prosthesis 2 will be subjected intraoperatively to thermal treatment.

In the embodiment of transport device 1 according to the invention depicted in the drawing, ossicular prosthesis 2 and associated prosthesis mock-up 3 are made of a material having memory effect, at least in the region of first fastening element 2a. The prosthesis can also comprise a device for varying the length thereof; in that particular case, the prosthesis and the associated prosthesis mock-up are made of a material having memory effect, at least in the region of the device for varying the length.

In many ossicular prosthesis and, optionally, the associated prosthesis mock-ups, a nickel-titanium alloy, Nitinol in particular, is used as the preferred material having memory effect.

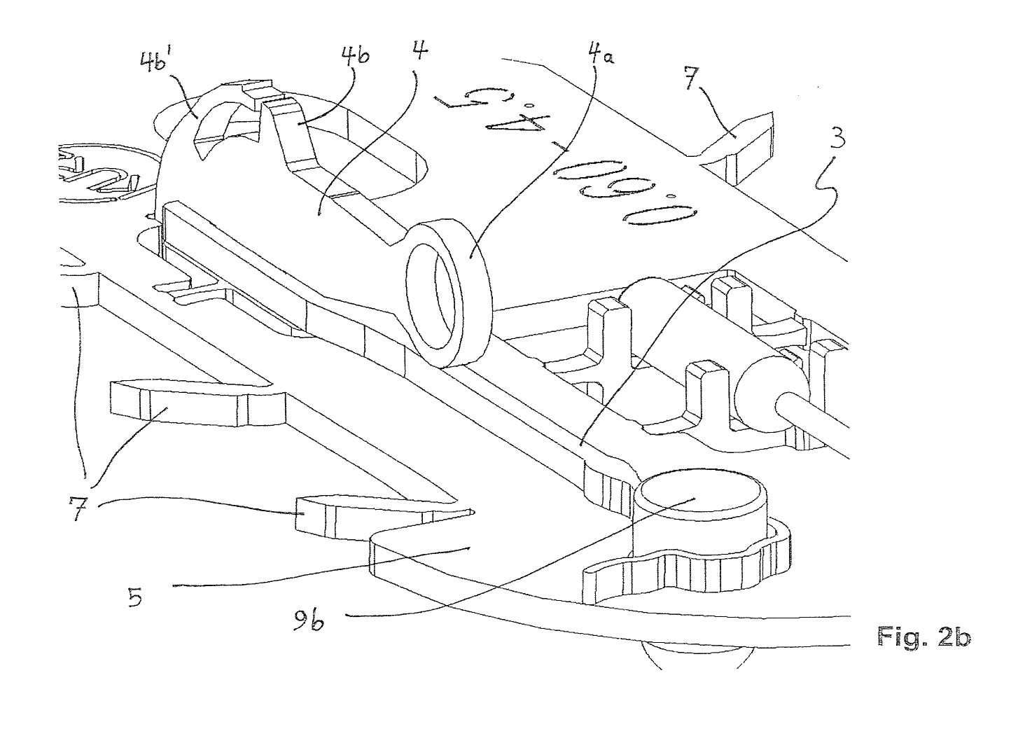

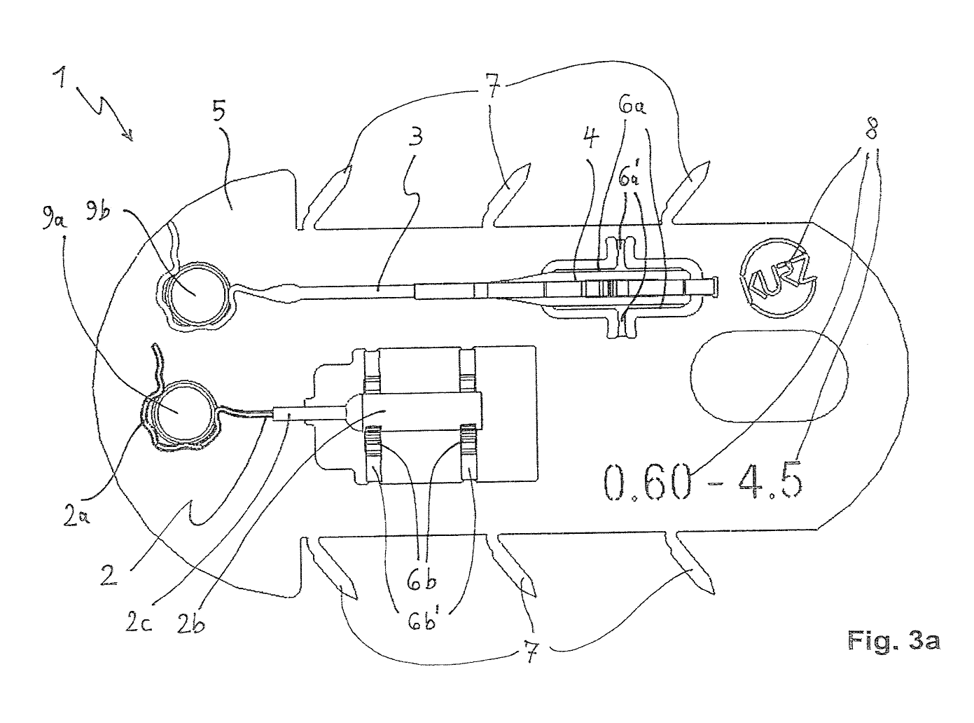

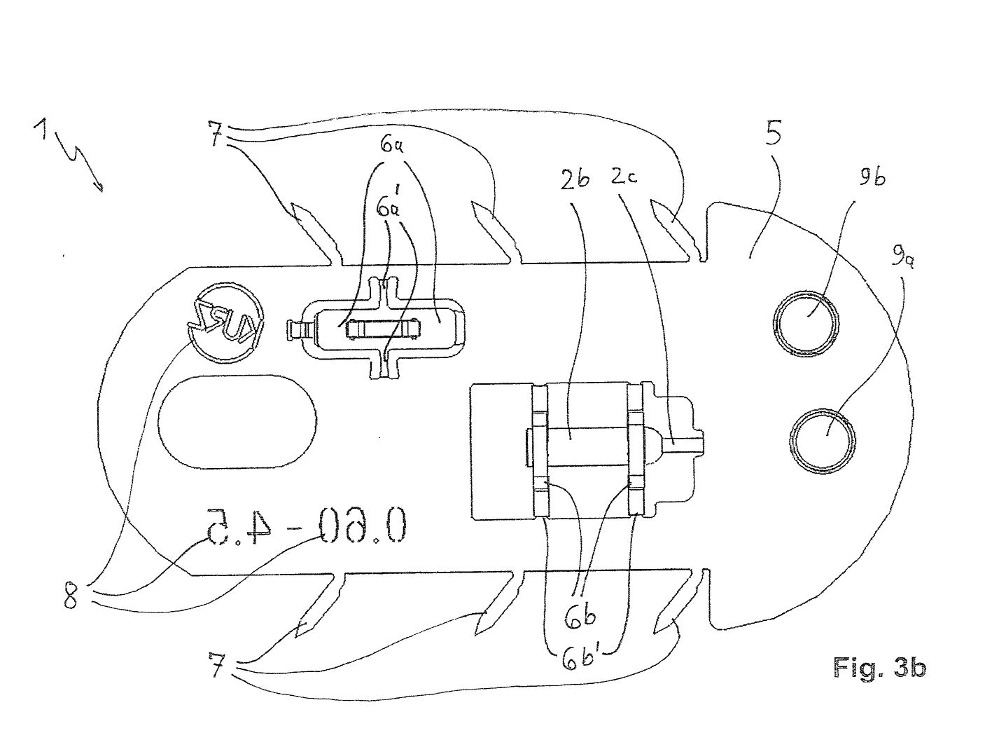

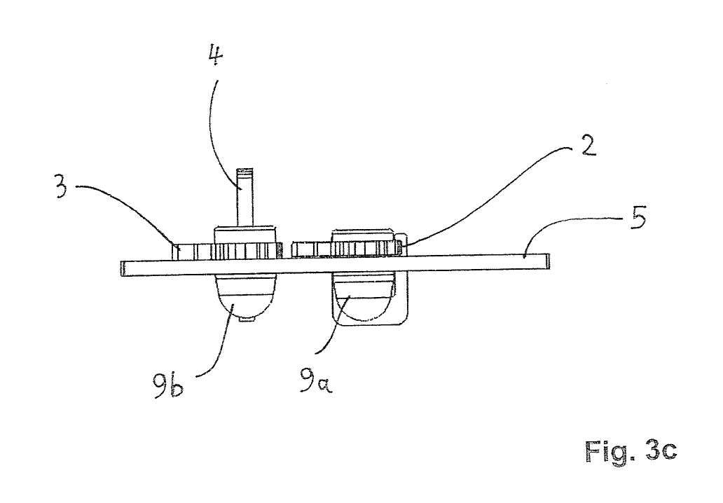

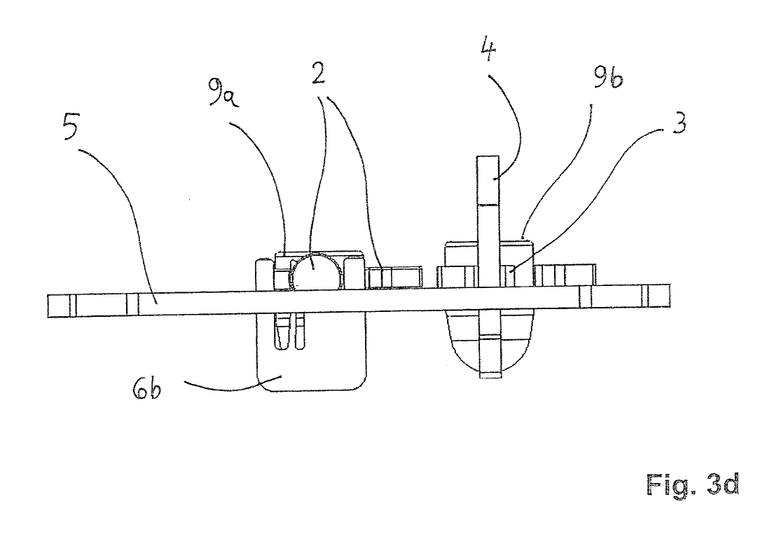

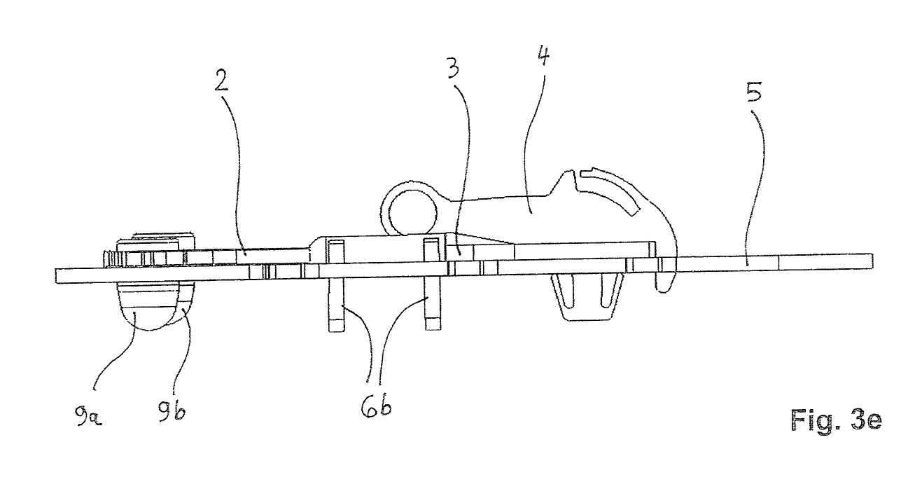

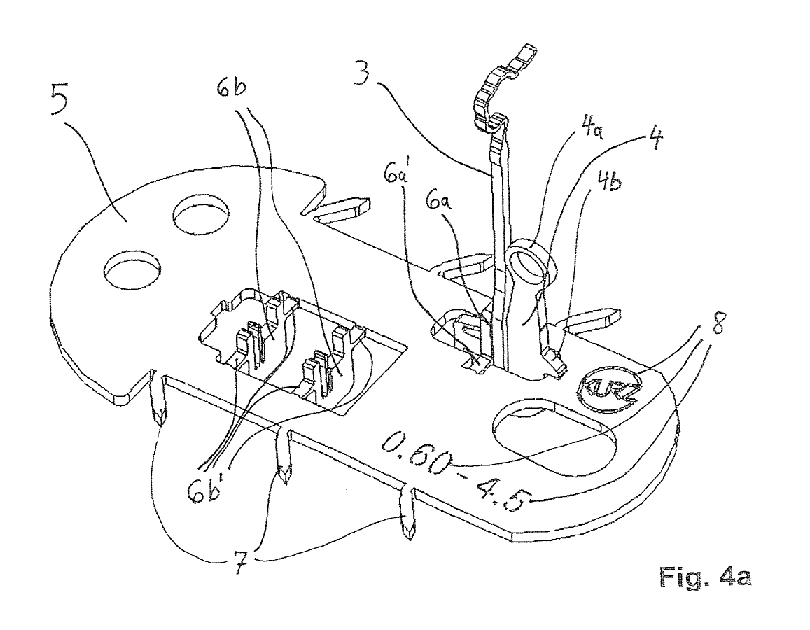

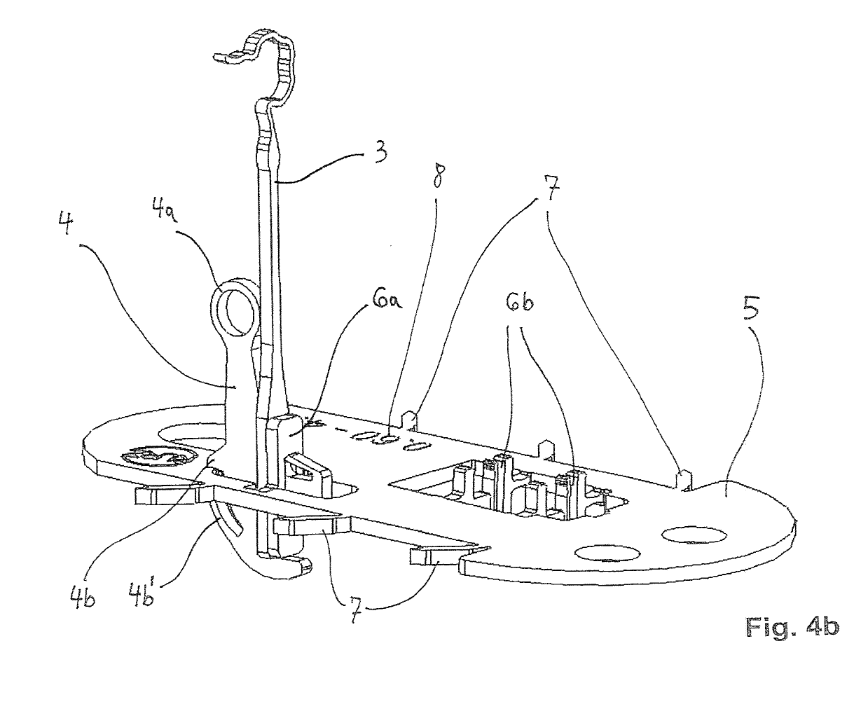

In every representation of prosthesis mock-up 3 shown in the drawing, a detent device 4 is provided, which is used to secure prosthesis mock-up 3 in a processing position which is preferably upright, as shown in FIGS. 4a and 4b. Possible details of detent device 4 are shown in enlarged depictions in FIGS. 2a and 2b, e.g. a tab-shaped operating element 4a or locking elements 4b, 4b′ which, in the locked state, enclose a counter-holder, as shown clearly in FIG. 4b.

A feature that is also common to all of the embodiments of transport device 1 according to the invention shown in the drawing is that a holding device which can swivellably accommodate prosthesis mock-up 3 is provided.

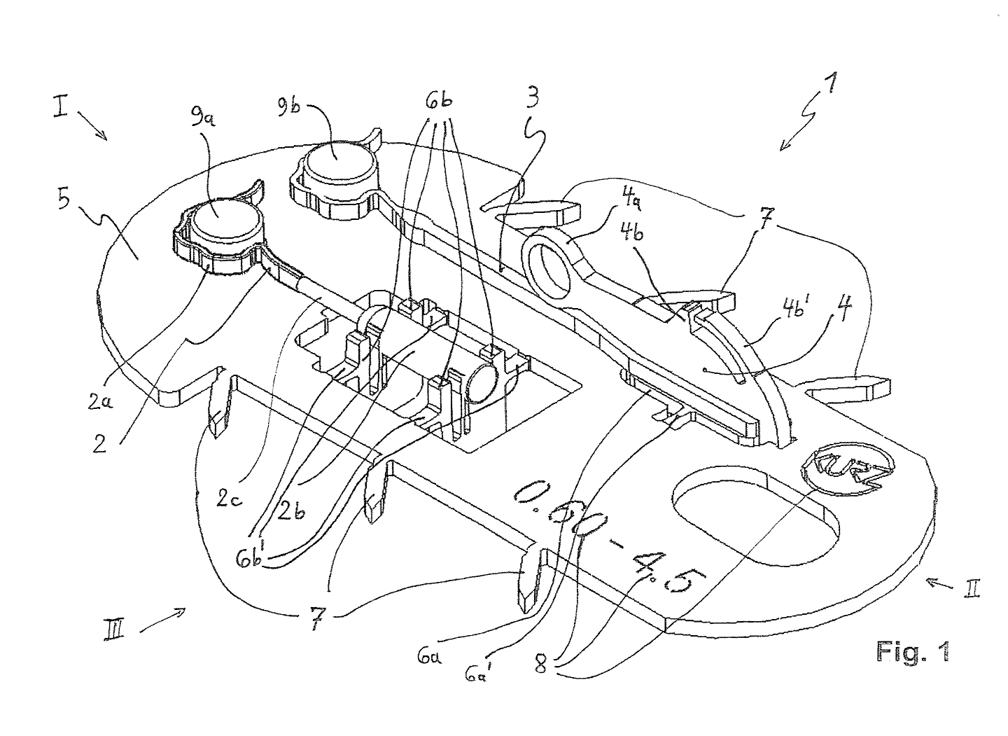

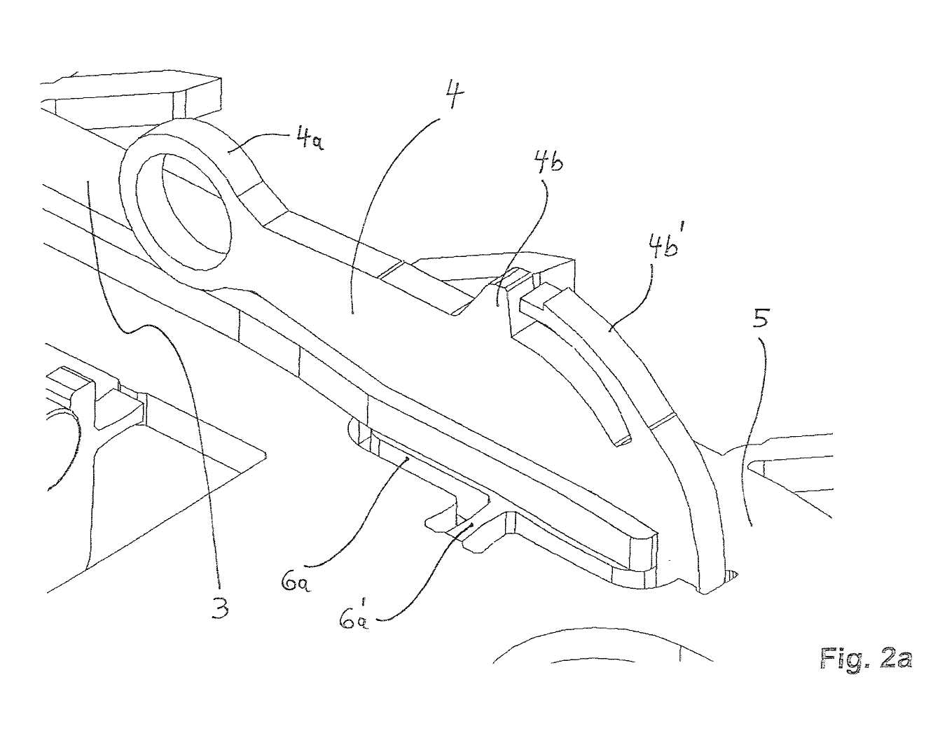

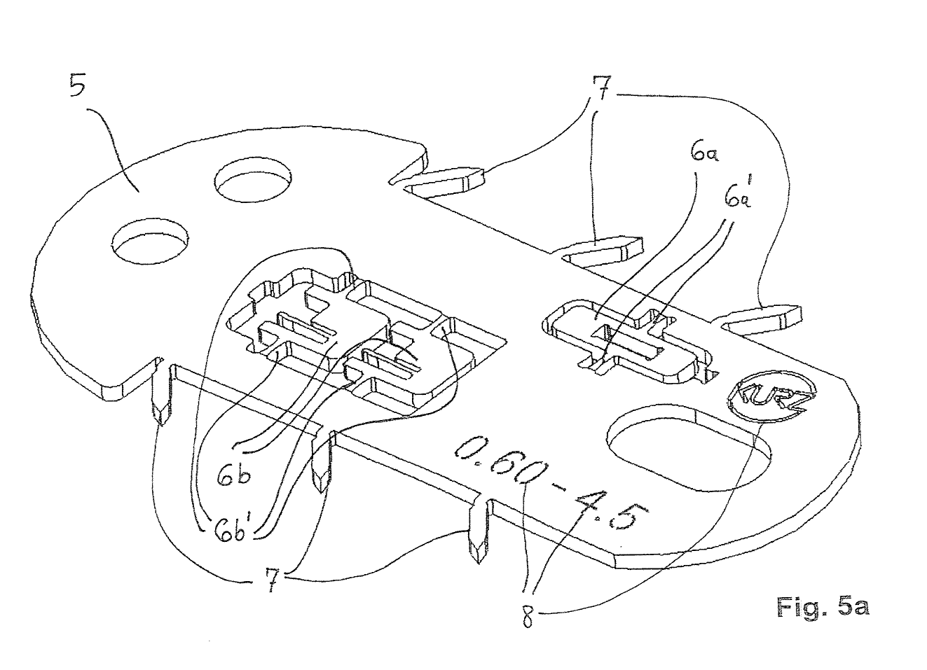

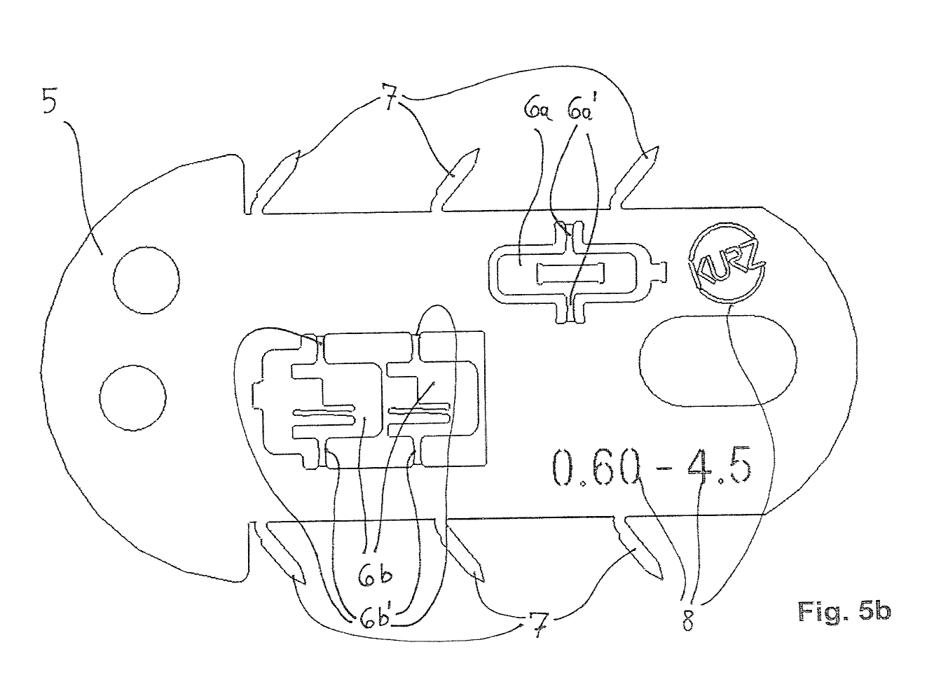

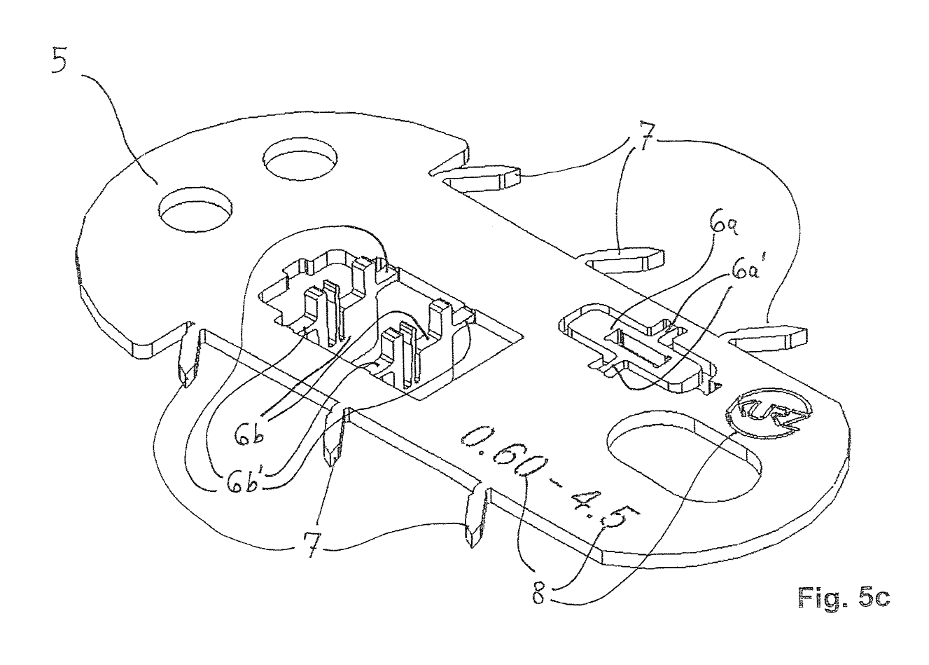

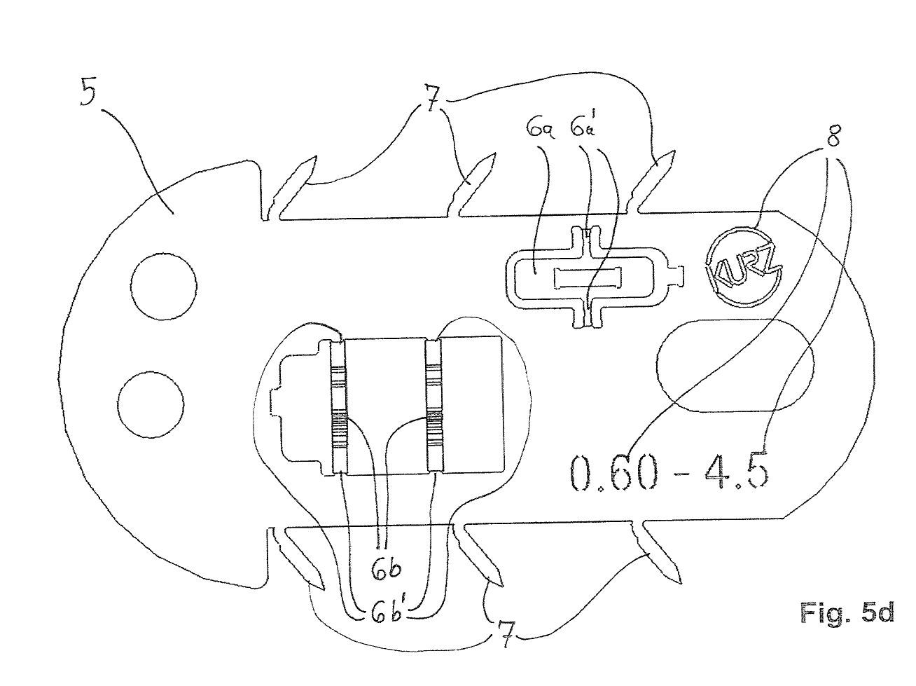

FIGS. 1 and 3a to 3e show base plate 5 with installed ossicular prosthesis 2 and prosthesis mock-up 3 in the lying-flat transport state; FIGS. 4a and 4b show prosthesis mock-up 3 upright and locked into position after removal of prosthesis 2; prosthesis mock-up 3 and prosthesis 2 are not present in FIGS. 5a to 5d; FIGS. 5a and 5b especially show the state of holding device 5 immediately after production thereof, and FIGS. 5c and 5d show the state shortly before installation of ossicular prosthesis 2 on holding device 5. Not shown are possible embodiments of the transport device according to the invention, in which the holding device is designed such that it can accommodate only the prosthesis mock-up, but not the associated ossicular prosthesis (or vice versa).

The figures of the drawing show a particularly preferred embodiment of the invention, in which the holding device for prosthesis mock-up 3 comprises a suspending device 6a which is connected to base plate 5 and can be twisted by way of fine segments 6a′, and is used to suspend one end of prosthesis mock-up 3. As mentioned above, FIGS. 5c and 5d illustrate, in particular, the state of transport device 1 shortly before installation of ossicular prosthesis 2, wherein suspending devices 6b for accommodating prosthesis 2 are rotated by way of segments 6b′ relative to the lying-flat state shown in FIGS. 5a and 5b by approximately 90° relative to base plate 5. In contrast, prosthesis mock-up 3 is suspended in the unrotated state of suspending device 6a for transporting the test device to the operating surgeon, and is swivelled by approximately 90° relative to the plate and locked into position—as shown clearly in FIGS. 4a and 4b—only when the optimal processing parameters of ossicular prosthesis 2 are to be determined.

Transport device 1 will typically include an outer packaging which encloses base plate 5 on all sides during transport, and is in the form of a container in particular, such as a case, a can, or a box, which is not depicted separately in the drawing.

Moreover, transport device 1 comprises a device for ensuring safe transport, which comprises burr-shaped segments 7 extending laterally outwardly from base plate 5 in the base-plate plane, which can also extend out of the base-plate plane at an angle in non-depicted embodiments of the invention. Segments 7 ensure that transport device 1—comprising installed ossicular prosthesis 2 which is relatively sensitive to mechanical damage, and prosthesis mock-up 3 which is also installed—will not slip during transport to the operating surgeon, wherein segments 7 can be clamped or braced against inner walls of the outer packaging mentioned above.

In the embodiment of the invention shown, base plate 5 is optically coded or labeled. The coding or labeling 8 contains technical information on ossicular prosthesis 2 and/or associated prosthesis mock-up 3, and manufacturer's information. The coding can also include simple color coding. As an alternative or in addition thereto—in embodiments which are not depicted in the drawing—the ossicular prosthesis and/or the associated prosthesis mock-up can also be optically coded and/or labeled, wherein the coding or labeling also contains technical information on the ossicular prosthesis and/or prosthesis mock-up.

The technical information in the coding or labeling 8 contains, for example, a size classification of ossicular prosthesis 2 used, and a recommended starting value for the electrical power or luminous power which should be applied within the scope of a test handling of prosthesis mock-up 3 at the beginning of the determination of optimized processing parameters for the parts of ossicular prosthesis 2 to be subjected intraoperatively to thermal treatment.

The coding and/or labeling 8 is created most simply using laser treatment and/or anodizing. Likewise, base plate 5 itself is preferably manufactured and machined further by laser-cutting the plate out of a larger sheet. In particular, the miniscule structures of the above-described elements of base plate 5, such as suspending devices 6a and 6b, associated twistable segments 6a′ and 6b′, and burr-shaped segments 7 for ensuring safe transport cannot be created in practically any other manner without expenditure becoming unreasonable.

In the embodiment shown of transport device 1 according to the invention, place-holder pins 9a, 9b are provided in base plate 5 to hold open first fastening element 2a of ossicular prosthesis 2, as well as the corresponding section of associated prosthesis mock-up 3, in a defined manner during transport to the operating surgeon; said place-holder pins 9a, 9b lock the thermally pretreated parts of ossicular prosthesis 2 composed of a material having memory effect, and optionally prosthesis mock-up 3, in the desired geometry, thereby ensuring that unwanted changes in shape are prevented even if warming occurs during transport.

The embodiment of transport device 1 according to the invention shown in the figures of the drawing comprises a middle ear prosthesis 2, in the case of which first fastening element 2a is in the form of a clamp which can be clipped onto the limb of incus, for example, or onto another component of the ossicular chain. In this embodiment, second fastening element 2b is designed, on the end opposite the clamp, as a piston for use to couple ossicular prosthesis 2 directly to the inner ear.

In embodiments of the invention that are not depicted in the drawing, the transport device can be designed such that it can accommodate an ossicular prosthesis, the fastening elements of which are designed with different geometries, e.g. as a sleeve, loop, or hook. The first fastening element can be a top plate which rests on the tympanic membrane. In addition, the second fastening element, for example, can be designed not as a piston but rather in the form of a clamp or piston for placement against the base of the stapes, or as a slotted bell for fastening the ossicular prosthesis to the stapes.

In further embodiments of the invention that are not depicted in the drawing, a ball joint can be integrated into connecting element 2c in order to ensure a certain amount of post-operative flexibility of ossicular prosthesis 2 between the connection points thereof. This is taken into account accordingly in the geometric design of suspending devices 6b.

In addition, the drawing does not show embodiments in which the ossicular prosthesis can be connected to an active vibration part of an active, partially implantable hearing aid.

It will be understood that each of the elements described above, or two or more together, may also find a useful application in other types of constructions differing from the types described above.

While the invention has been illustrated and described as embodied in a transport device for ossicular prosthesis, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A transport device for receiving and securely holding an ossicular prosthesis for implantation in a middle ear, during transport to an operating surgeon for replacing or bridging at least parts of a component of a human ossicular chain, the transport device comprising a flat base plate of thin metal sheet having a thickness of substantially half a millimeter; at least one suspending device connected to said base plate and twistable using fine segments and integrated in said base plate for receiving and holding the ossicular prosthesis; and a connecting element which connects a first fastening element of the ossicular prosthesis used for mechanical connection to a tympanic membrane or a component of the ossicular chain, with a second fastening element on another end used for mechanical connection to a further component or parts of a component of the ossicular chain or directly to an inner ear so that the first and second fastening elements are connected by said connecting element in a sound-conducting manner, wherein said suspending device is configured so that it aligns with a surface of said base plate during manufacture of said transport device and for suspension of the ossicular prosthesis is rotatable relative to an ossicular prosthesis lying-flat production state by substantially 90° relative to said base plate by plastic deformation of said segments and then extends above the surface of said base plate.

2. The transport device as defined in claim 1, wherein said base plate is configured as a plate machined using a process selected from the group consisting of a laser treatment, an anodizing, and both.

3. The transport device as defined in claim 1, wherein said base plate and said suspending device are formed as components of one integrated blank.

4. The transport device as defined in claim 1, further including an outer packaging enclosing said base plate on all sides during transport.

5. The transport device as defined in claim 4, further comprising a device for ensuring safe transport and provided with burr-shaped segments extending laterally outwardly from said base plate and engagable against inner walls of said outer packaging in a manner selected from the group consisting of clamped and braced against said inner walls of said outer packaging.

6. The transport device as defined in claim 1 for the ossicular prosthesis having at least parts made of a material having memory effect, thermally pretreated, and subjected to a further shape-altering treatment parameter when the ossicular prosthesis is implanted in the middle ear, wherein the device is provided for determining a parameter selected from the group consisting of an optimized power setting, an energy application site for preparing parts of the ossicular prosthesis to be subjected intraoperatively to thermal treatment, and both, and contains a prosthesis mock-up which is configured to match the ossicular prosthesis and to be identical to the ossicular prosthesis in terms of material, geometric shape, and manufacturing method, and further comprising a holding device which swivellably accommodates said prosthesis mock-up.

7. The transport device as defined in claim 6, wherein said holding device for said prosthesis mock-up has a suspending device which is connected to said base plate and is twistable by said segments, and is used to suspend one end of said prosthesis mock-up.

8. The transport device as defined in claim 6, wherein said prosthesis mock-up contains a detent device usable to secure said prosthesis mock-up on said base plate at least in one processing position.

9. The transport device as defined in claim 6, further comprising place-holder pins associated with said base plate in a manner selected from the group consisting of inserted into said base plate and integrated in said base plate, said place-holder pins being formed such that they affix the thermally treated parts of the ossicular prosthesis composed of material having memory effect, and optionally corresponding sections of said prosthesis mock-up in a desired geometry during transport to the operating surgeon such that unwanted changes in shape are prevented even if warming occurs during transport.

10. The transport device as defined in claim 6, wherein said base plate is formed as a base plate selected from the group consisting of an optically coded base plate, a labeled base plate, and both, wherein a coding or labeling of said base plate contains technical information selected from the group consisting of technical information regarding the ossicular prosthesis, technical information regarding the mock-up, technical information regarding manufacturer's information, and combinations thereof.

11. The transport device as defined in claim 10, wherein said technical information regarding the coding or labeling on said base plate contains a recommended starting value for a power selected from the group consisting of an electrical power, a luminous power which should be used within the scope of a test handling of said mock-up at a beginning of a determination of optimized processing parameters for components of the ossicular prosthesis to be subjected intraoperatively to thermal treatment.

12. The transport device as defined in claim 1, wherein said base plate is configured as a working plate for preparation of the ossicular prosthesis before implantation in the middle ear.

13. The transport device as defined in claim 12, wherein said base plate is configured as a working plate for setting a specific desired length of the ossicular prosthesis whose length is variable.

14. The transport device as defined in claim 4, wherein said outer packaging is in a form of a container selected from the group consisting of a case, a can, and a box.
